(19) United States
(12) Patent Application Publication
(10) Pub. No.: US 2011/0009695 A1
(43) Pub. Date: Jan. 13, 2011

(54) BENDING SECTION FOR ENDOSCOPE

(76) Inventor: Sung Yong Lee, Gangwon-do (KR)

Correspondence Address: LADAS & PARRY LLP, 224 SOUTH MICHIGAN AVENUE, SUITE 1600, CHICAGO, IL, 60604, US

(21) Appl. No.: 12/667,457

(22) PCT Filed: Jun. 30, 2008
(86) PCT No.: PCT/KR2008/003838

§ 371 Date: Apr. 14, 2010

(30) Foreign Application Priority Data
Jul. 3, 2007 (KR) 10-2007-0066548

(51) Int. Cl. A61B 1/008; A61B 1/04; H04N 7/18

(52) U.S. Cl. 600/109; 600/141; 348/75; 348/E07.085

(57) ABSTRACT

A bending device for an endoscope having a camera mounted on one end for photographing the inside of a human body, a machine, a pipe and the like is provided. For flexible bending with the smallest external diameter and the largest extended internal diameter, a plurality of bending plates each having an internal hole (11) are stacked so that the bending device can be bent, each bending plate including a pair of upper protruded portions (12) curved upward at opposite locations; a pair of lower protruded portions (13) formed between the upper protruded portions (12) and curved downward; and adjustment holes (14) punched in the upper protruded portions (12) or the lower protruded portions (13) so that the adjustment wires (20) pass through the adjustment holes (14), and the bending plates are cross-stacked so that the upper protruded portions (12) and the lower protruded portions (13) of the adjacent bending plates are brought into contact with each other. Thus, sufficient promptness, convenience, and flexibility for inside photographing can be achieved.

### TECHNICAL FIELD

The present invention relates to a bending section for an endoscope that enables a camera device to be freely bent in desired directions in an endoscope for enabling a user to image and observe a narrow internal space of a human body or a machine.

### BACKGROUND ART

In a medical industry, an endoscope enables a user to observe the inside of a human body (e.g., stomach, bronchus, esophagus, large intestine, small intestine, etc.) using a small camera and check abnormality thereof without cutting the human body, for example, through surgery or autopsy. In recent years, the endoscope is widely used in a variety of industries, including the medical industry, to observe the inside of a precise machine without disassembling it or observe abnormality of the inside of pipes.

A typical endoscope apparatus has a camera mounted at an end of a bending device, which includes an image data transfer wire for transferring an image photographed by the camera, a power supply wire for supplying power to the camera, and adjustment wires for adjusting bending of the bending device.

To use the endoscope apparatus having such a structure, a user inserts the camera into a specific part of a human body or an observation destination of a machine, and pulls the adjustment wires to change or adjust a photographing direction or angle of the camera when the camera arrives at a proper location.

However, in this endoscope, a bending mechanism for adjusting the photographing angle is costly because of its complex structure and precision requirement and is highly likely to malfunction. For example, the bending mechanism includes a plurality of pipes interconnected by connection members, such as pins, and bending gears.

Since bending members are mounted inside the bending device and the power supply wire is installed outside the bending device, the wire may be damaged and the bending direction or angle of the bending device for changing the photographing angle of the camera is limited. As a result, the bending device has low durability and makes precise photographing difficult.

### DISCLOSURE OF INVENTION

### Technical Problem

The present invention provides a bending device for an endoscope that has promptness for rapid driving with various wires inserted into the bending device being included in a sufficiently extended internal hole in a stable state, has such a high flexibility that a bending direction or angle can be freely adjusted and controlled, and has a simple and inexpensive structure.

Additional features of the invention will be set forth in the description which follows, and in part will be apparent from the description, or may be learned by practice of the invention.

### Technical Solution

The present invention discloses a bending device for an endoscope, wherein a camera is mounted on one end of the bending device and a plurality of bending plates each having an internal hole 11 are stacked so that the bending device can be bent and each bending plate comprises: a pair of upper protruded portions 12 curved upward at opposite locations; a pair of lower protruded portions 13 formed between the upper protruded portions 12 and curved downward; and adjustment holes 14 punched in the upper protruded portions 12 or the lower protruded portions 13 so that adjustment wires 20 pass through the adjustment holes 14, and wherein the bending plates are cross-stacked so that the upper protruded portions 12 and the lower protruded portions 13 of the adjacent bending plates are brought into contact with each other.

In the present invention, the bending plate may have any shape only if it has an internal hole 11 having a sufficient internal diameter so that various wires are inserted into the bending plate. For smoother bending, the bending plate may be in any one of circular, elliptic, lozenge, and rectangular shapes. Preferably, the bending plate may be in a circular shape.

It is to be understood that both the foregoing general description and the following detailed description are exemplary and explanatory and are intended to provide further explanation of the invention as claimed.

### Advantageous Effects

According to the present invention, the bending device for an endoscope has an internal hole having a sufficient size so that a photographing data transfer wire, a power supply wire and the like are disposed inside the bending device, and has a minimized external diameter for promptness and miniaturization such that the bending device can be rapidly driven in a stable state and used optimally with improved promptness in any environments.

Furthermore, the bending device is flexible so that it can be freely bent in two or four directions by adjusting the adjustment wires disposed inside the bending device. This allows for easy observation irrespective of the location of a subject. In addition, the bending device is less likely to malfunction because of its simple structure.

### BRIEF DESCRIPTION OF THE DRAWINGS

The accompanying drawings, which are included to provide a further understanding of the invention and are incorporated in and constitute a part of this specification, illustrate embodiments of the invention, and together with the description serve to explain the principles of the invention.

FIG. 1 is an exploded perspective view illustrating a bending device for an endoscope according to an embodiment of the present invention;

FIG. 2 is a cross-sectional view illustrating a bending device for an endoscope in a bending state according to an embodiment of the present invention; and

FIG. 3 is a perspective view illustrating bending directions and use of a bending device for an endoscope according to an embodiment of the present invention.

### MODE FOR THE INVENTION

The invention is described more fully hereinafter with reference to the accompanying drawings, in which exemplary embodiments of the invention are shown. This invention may, however, be embodied in many different forms and should not be construed as limited to the embodiments set forth herein. Rather, these embodiments are provided so that this disclosure is thorough, and will fully convey the scope of the invention to those skilled in the art.

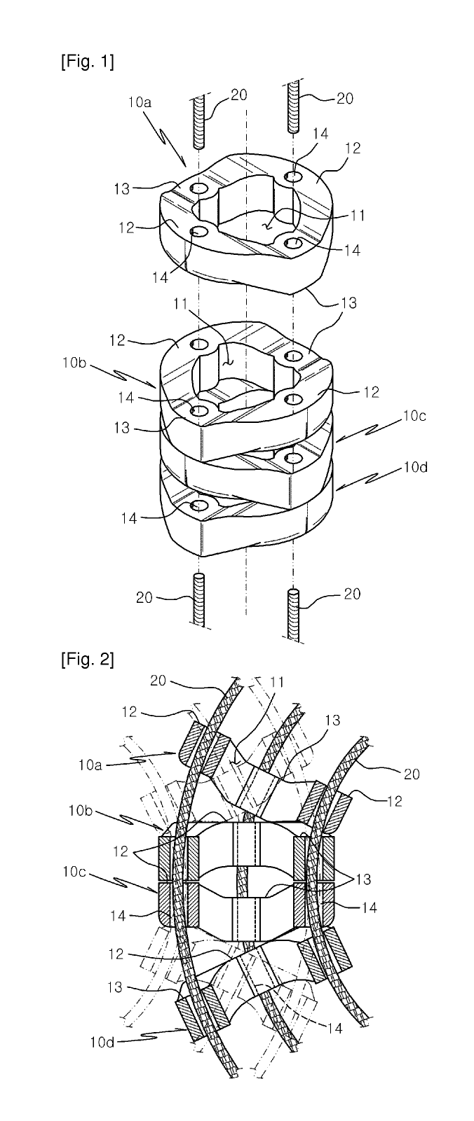

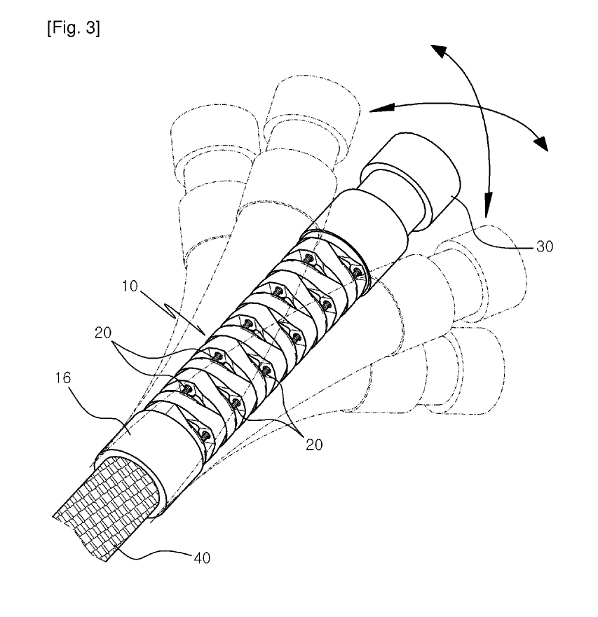

FIG. 1 is an exploded perspective view illustrating a bending device for an endoscope according to an embodiment of the present invention, FIG. 2 is a cross-sectional view illustrating a bending device for an endoscope in a bending state according to an embodiment of the present invention, and FIG. 3 is a perspective view illustrating bending directions and use of a bending device for an endoscope according to an embodiment of the present invention.

Referring to FIG. 3, in a bending device 10 for an endoscope according to the present invention, a camera 30 is mounted at one end of the bending device for photographing a subject, and a tube 40 is connected to the other end for accepting and guiding various wires.

The various wires may include, for example, an image data transfer wire for transferring image data from the camera, a power wire for supplying power to the camera, and/or adjustment wires 20 for adjusting a bending direction or angle of the bending device 10.

The bending device 10 can be bent in four directions of forward/backward/left/right to change the direction or angle so that the camera can photograph a desired location. As shown in FIGS. 1 and 2, a plurality of bending plates 10a, 10b, 10c and 10d are stacked such that the bending device can be bent.

The bending device 10 may be bent only in two directions, such as forward/backward directions, or in more directions rather than in the four directions shown, if necessary. A structure of the bending device 10 that can be bent in two or four directions will be described in detail with reference to FIGS. 1 and 2.

Referring to FIGS. 1 and 2, each of the plurality of bending plates 10a, 10b, 10c and 10d has an internal hole 11 having a sufficient internal diameter for various wires to be inserted into the bending device. Each bending plate has a circular, lozenge, elliptic, or rectangular shape, but is not limited thereto. In this embodiment, each bending plate has a circular shape with a predetermined thickness, as shown.

Each of the bending plates 10a, 10b, 10c and 10d has a predetermined curved surface. As shown, the bending plate has a pair of upper protruded portions 12 curved upward and protruded upward at opposite locations, and lower protruded portions 13 formed between the pair of upper protruded portions 12 and at opposite locations.

The lower protruded portions 13 are continuously curved downward from the upper protruded portions 12, such that the lower protruded portions 13 are relatively lower in height than the upper protruded portions 12. The upper protruded portions 12 and the lower protruded portions 13 are disposed in a 90° direction and at the opposite locations.

Meanwhile, the upper protruded portions 12 and the lower protruded portions 13 have adjustment holes 14 through which the adjustment wires 20 pass. When the adjustment wires 20 pass through the adjustment holes 14, the adjacent bending plates 10a, 10b, 10c and 10d are cross-stacked.

That is, the lower protruded portions 13 of the first bending plate 10a are brought into contact with the upper protruded portions 12 of the second bending plate 10b, and the lower protruded portions 13 of the second bending plate 10b are brought into contact with the upper protruded portions 12 of the third bending plate 10c.

In thin structure, when any of the wires is pulled as shown in FIG. 2, the right upper protruded portion 12 of the first bending plate 10a is brought into contact with the right lower protruded portion 13 of the second bending plate 10b while the left upper protruded portion 12 of the first bending plate 10a is got away from the left lower protruded portion 13 of the second bending plate 10b. The same applies to the four bending plate 10d and the third bending plate 10c.

When several bending plates are stacked in this manner, the bending device 10 can be bent by pulling any of the adjustment wires 20. It will be easily understood from this disclosure and the drawings that, where the bending plates 10a, 10b, 10c and 10d are stacked, the bending device 10 can be bent as an interval between the upper protruded portions and the lower protruded portions of the adjacent bending plates is increased or decreased by the adjustment wires 20.

When the adjustment holes 14 are formed at only two opposite locations rather than four positions or two adjustment wires 20 are disposed in the adjustment holes 14 formed at four positions, the bending device 10 can be bent in only two directions at which the adjustment wires 20 are disposed. This structure variation may be selected according to a user or manufacturer's need.

For a large bending angle of the bending device 10, a height difference between the upper protruded portions 12 and the lower protruded portions 13 of the bending plates 10a, 10b, 10c and 10d may increase or the number of the bending plates may increase.

It will be apparent to those skilled in the art that various modifications and variations can be made in the present invention without departing from the spirit or scope of the invention. Thus, it is intended that the present invention cover the modifications and variations of this invention provided they come within the scope of the appended claims and their equivalents.

1. A bending device for an endoscope, wherein a camera is mounted on one end of the bending device and a plurality of bending plates each having an internal hole 11 are stacked so that the bending device can be bent and each bending plate comprises:
a pair of upper protruded portions 12 curved upward at opposite locations;
a pair of lower protruded portions 13 formed between the upper protruded portions 12 and curved downward; and
adjustment holes 14 punched in the upper protruded portions 12 or the lower protruded portions 13 so that adjustment wires 20 pass through the adjustment holes 14, and
wherein the bending plates are cross-stacked so that the upper protruded portions 12 and the lower protruded portions 13 of the adjacent bending plates are brought into contact with each other.

2. The device of claim 1, wherein the adjustment holes 14 are punched in both the upper protruded portions 12 and the lower protruded portions 13 of the respective bending plates, and the respective adjustment wires 20 pass through the opposite adjustment holes 14.

3. The device of claim 1 or 2, wherein the bending plate is in any one of circular, elliptic, lozenge, and rectangular shapes.
